(19) United States
(12) Patent Application Publication
(10) Pub. No.: US 2007/0015177 A1
(43) Pub. Date: Jan. 18, 2007

(54) Nucleic acid extraction method

(75) Inventors: Pierre-Alain Maron, Dijon (FR); David Lejon, Dijon (FR); Karine Bizet, Boulogne-Billancourt (FR); Esmeralda Carvalho, Arnouville (FR)

Correspondence Address: BIRCH STEWART KOLASCH & BIRCH, PO BOX 747, FALLS CHURCH, VA, 22040-0747, US

(73) Assignee: BERTIN TECHNOLOGIES, Montigny-Le-Bretonneux (FR)

(21) Appl. No.: 11/399,420

(22) Filed: Apr. 7, 2006

Related U.S. Application Data
(63) Continuation of application No. PCT/FR04/02613, filed on Oct. 13, 2004.

(30) Foreign Application Priority Data
Oct. 15, 2003 (FR) 0312049

(51) Int. Cl. C12Q 1/68; C12N 1/08; C07H 21/04

(52) U.S. Cl. 435/6; 435/270; 536/25.4

(57) ABSTRACT

The invention relates to a method for the extraction of nucleic acids from microorganisms and to the use thereof in a method for the analysis of a microbial population in a given environment, e.g. a population of microorganisms taken from the air. In particular, the invention relates to an extraction method in which the following three lysis steps are performed successively in any order, namely: (i) chemical lysis of the microorganisms, consisting in bringing the microorganisms into contact with a lysis solution containing a detergent; (ii) thermal shock lysis comprising the incubation of the extract at a temperature lower than 0° C., immediately followed by the incubation of the extract at a temperature of at least 95° C. and preferably 100° C.; and mechanical lysis in which the extract is stirred in the presence of balls.

The present invention relates to a method for extracting nucleic acids from microorganisms and to its use in a method for analyzing a microbial population from a given environment, in particular a population of microorganisms sampled from air. In particular, the invention pertains to an extraction method comprising successive implementation of the following three lysis steps in any order:
i. chemical lysis of microorganisms, comprising bringing microorganisms into contact with a lysis solution containing a detergent;
ii. heat shock lysis, comprising incubating the extract at a temperature below 0° C., immediately followed by incubating the extract at a temperature of at least 95° C., preferably about 100° C.; and
iii. mechanical lysis, comprising agitating the extract in the presence of beads.

Climate changes or changes to the ecosystem associated with urbanization, agriculture or industry are generally correlated with modifications in the composition of the microbial flora in the environment. Thus, in theory it is possible to detect abnormal modifications to the ecosystem by analyzing the change in the composition of a microbial population of a given environment over time.

Such monitoring would also be useful in detecting the abnormal presence of pathogenic agents in the environment, for example linked to their dissemination in air by biological weapons.

However, the impossibility of cultivating the majority of microorganisms sampled from the environment such as earth or air constitutes a major obstacle to analyzing microbial ecology and its diversity.

Such an obstacle may be overcome using molecular biological techniques, by direct analysis of nucleic acid sequences specific to microorganism species contained in the analyzed sample, thereby avoiding culture steps.

In particular, the RISA (ribosomal intergenic spacer amplification) technique based on an analysis of the length polymorphism in the 16S/23S intergenic region of ribosomal RNA, allows populations of microorganisms to be characterized and allows them to be compared (Ranjard et al, October 2001, Applied and Environmental Microbiology, pp 4479-4487, Ranjard et al, 1999, FEMS Microbiology Ecology, 0168-6496, Ranjard et al, 2000, Applied and Environmental Microbiology, vol 66, pp 5334-5339; Ranjard et al, 2000, Microbial Ecology, vol 39 (4), pp 263-272, Selenska-Pobell et al, 2001, Antonie van Leeuwenhoek, vol 79 (2), pp 149-161).

Those techniques, based on an analysis of genomic sequences, generally use PCR amplification of nucleic acids to obtain a sufficient quantity of analyzable genetic material.

However, in order to be sufficiently sensitive and quantitative, the amplification technique per se must be carried out starting from a sufficiently large quantity of genetic material extracted from the sample.

That quantity of genetic material extracted from the sample clearly depends firstly on the quantity of microorganisms sampled, and secondly on the yield of the method for extracting nucleic acids used and the purity obtained. It appears that the method for extracting nucleic acids is a determining factor in obtaining reliable results starting from a method for analyzing a microbial population based on analysis of nucleic acid sequences extracted from microorganisms of a sample.

Many methods for extracting nucleic acids from microorganisms are known in the art. Such methods generally comprise a lysis step, consisting of rupturing the bacterial or fungal wall and membrane, a step for precipitating membrane and protein debris, the nucleic acid remaining in solution, then a step for precipitating nucleic acids in alcohol, possibly preceded by a step for purifying nucleic acids in the presence of phenol and chloroform. Such extraction techniques are well known to the skilled person and have in particular been described in Sambrook et al (Molecular Cloning: A Laboratory Manual, 3^{rd} edition, Cold Spring Harbor, Laboratory Press, Cold Spring Harbor, N.Y.). Optimized protocols for extracting DNA from microorganisms removed from the environment, such as those described by Yeates et al, 1998 (Biological procedures, 1998, vol 1, No 1, pages 40-47), can in particular be cited.

However, such extraction techniques are still insufficient as regards yield or quality when used in certain microbial analysis procedures.

While certain environmental media, such as water or earth, are rich in microorganisms, this is not the case when analyzing other environments such as air, which requires sampling from very large volumes, in particular because the extraction yields are insufficient. High extraction yields are also required when the sampled microorganisms are in the form of spores, which are naturally more resistant to lysis steps.

As a result, there is a genuine need for the development of novel methods for extracting nucleic acids in high yields, which could be used routinely in methods for analyzing the microbial population of the environment.

The first step in the nucleic acid extraction methods consists in lysing microorganism membranes. Known extraction protocols employ three alternative modes of lysis: chemical lysis using a detergent, mechanical lysis by agitation in the presence of beads, and heat shock lysis by repeated freezing and incubation at very high temperature.

While the heat shock or mechanical lysis steps are generally considered to be alternatives to chemical lysis, it has now surprisingly been established that the successive implementation of three steps of chemical, mechanical lysis and heat shock lysis could considerably increase the extraction yield, in particular in comparison with a combination of only two of the three lysis steps.

A first aim of the invention thus pertains to a method for extracting nucleic acids from microorganisms, comprising:
a. successive implementation of the following three lysis steps in any order:
i. chemical lysis of microorganisms, comprising bringing microorganisms into contact with a lysis solution containing a detergent;
ii. heat shock lysis, comprising incubating the extract at a temperature below 0° C., immediately followed by incubating the extract at a temperature of at least 95° C., preferably about 100° C.; and
iii. mechanical lysis, comprising agitating the extract in the presence of beads;
b. precipitating membrane and protein debris;
c. recovering the supernatant containing the nucleic acids;
d. precipitating the nucleic acids and eliminating the supernatant;
e. if appropriate, taking the nucleic acids up into solution in a suitable buffer.

According to the method of the invention, the microorganisms are taken up into suspension in a solution containing the appropriate lysis buffer for carrying out one of the three lysis steps: chemical, mechanical or thermal.

In the text below, the term “extract” will be used to designate the solution containing the nucleic acids to be extracted obtained after any one of the steps of the extraction method, from taking the microorganisms into suspension before their lysis to the final extract obtained.

Unless otherwise indicated, the term “nucleic acids” will be used to designate deoxyribonucleic acids (DNA), single or double strand, or ribonucleic acids (RNA), including in particular messenger RNA, ribosomal RNA and transfer RNA.

In the context of the invention, the chemical lysis step comprises bringing microorganisms into the presence of a solution containing a sufficient quantity of detergent determined by the skilled person to obtain an optimum yield. Examples of detergents which can be used for chemical lysis are CTAB or sodium dodecyl sulfate (SDS). In a specific implementation, SDS is used as the detergent, for example in a concentration in the range 1% to 4%, conventionally about 2%.

The lysis step by heat shock comprises incubating the extract at a temperature of less than 0° C., immediately followed by incubating the extract at a temperature of at least 95° C., preferably about 100° C.

The incubation temperatures are selected so that the temperature of the extract reaches a value which is below 0° C. then a value which is above 95° C. These are, for example, in the range −90° C. to 0° C. for the low point, preferably in the range −80° C. to −60° C., and in the range 95° C. to 110° C. for the high point, preferably in the range 95° C. to 100° C. The heat shock means that the temperature of the extract changes from a temperature of less than 0° C. to a temperature of more than 95° C. in less than 1 minute, preferably less than 30 seconds, and more preferably in less than 1 second.

Conventionally, for a volume of extract of less than 1 millilitre, the extract is incubated at a temperature of less than 0° C. until it freezes, then incubated at a temperature of more than 95° C., preferably about 100° C., for at least 2 minutes, preferably 3 minutes.

To freeze the extract rapidly, liquid nitrogen may be used or any other inert gas which is liquid at a temperature of much lower than 0° C.

Further, in a particular implementation, heat shock lysis comprises incubating the extract at a temperature of about −70° C., for example in liquid nitrogen, immediately followed by incubating the extract at a temperature of at least 95° C., preferably about 100° C., for example in boiling water.

The heat shock described above may be repeated as many times as is necessary, preferably at least three times, to obtain an optimum yield.

If appropriate, the lysis step may be preceded by enzymatic lysis to degrade the proteins of the microbial walls, for example by incubating the microorganisms in suspension in a solution containing proteases at a suitable temperature. Examples of proteases which may be cited are K proteinases, conventionally used in protocols for extracting nucleic acids.

In accordance with the invention, mechanical lysis comprises agitating the extract in the presence of beads. The rate of agitation, the bead size and the quantity of beads used are determined by the skilled person so as to weaken the membranes in an optimum manner. The beads are small diameter glass beads which are conventionally used in protocols for extracting nucleic acids by mechanical lysis, in particular protocols for extracting nucleic acids of microorganisms comprising a rigid wall.

In particular, the extract may be agitated using a mill, in the presence of beads with a diameter in the range 50 μm to 200 μm, preferably in the range 90 μm to 150 μm, for example about 100 μm. Beads with different diameters may be combined. In particular, beads with a diameter in the range 50 μm to 200 μm, in an amount of 500 mg, for example between 400 mg and 600 mg, for a volume of extract in the range 500 to 1500 μl, may be used in combination with several larger diameter beads, for example 1 to 10 beads with a diameter in the range 1 to 3 mm, for example 4 beads 2 mm in diameter.

Preferably, the three lysis steps defined in (a) are carried out in the following order: (i) chemical lysis, (ii) heat shock lysis and (iii) mechanical lysis.

Once the lysate has been obtained, it is possible to carry out further enzymatic degradation of the proteins contained in the lysate, for example using proteases such as K proteinases.

The supernatant containing the nucleic acids in solution is then recovered and the nucleic acids are precipitated using conventional methods, for example in the presence of a salt such as sodium acetate and alcohol, in particular ethanol or isopropanol. If necessary, the precipitation step may be followed by a purification step, for example using a mixture of phenol and chloroform.

After precipitation, the nucleic acids are taken up into solution in a suitable buffer.

The nucleic acid extraction method is suitable for extracting nucleic acids from any type of prokaryotic or lower eukaryotic microbial population, including bacteria, protozoa, single celled algae, archaebacteria or fungi.

More particularly, the method of the invention allows nucleic acids to be extracted from microbial organisms which are principally bacteria and/or fungi.

These microbial organisms may in particular comprise spores of bacteria and/or fungi.

In a particular implementation, the lysis steps are preceded by a step for culturing spores under conditions appropriate to initiate germination, for example by incubating spores in a rich culture medium at a suitable temperature, conventionally for between 15 minutes and 3 hours at the optimum culture temperature for vegetative forms of microorganisms, for example 15 to 45 minutes at 37° C.

The microorganisms may be removed from any type of natural environment, including dirt, water or air. In particular, the method is suitable for removing microorganisms contained in air.

The method is also suitable for extracting nucleic acids from a limited number of microorganisms, for example in the range 103 to 10⁸ CFU/ml, preferably in the range 10⁶ to 10⁷ CFU/ml.

Because of the high yields obtained by the nucleic acid extraction method of the invention, said method is particularly suitable for use in a method for analyzing a microbial population.

In a second aspect, then, the invention pertains to a method for analyzing the microbial population of an environment, comprising:
a. removing a sample of the microbial population from the environment, for example in the form of an aerosol, and taking the sample up into suspension in a solution;
b. extracting nucleic acids from the microbial population in suspension using the extraction method of the invention as defined above;
c. analyzing the extracted nucleic acids.

In a specific implementation of the method, the microbial population to be analyzed is sampled from air. In particular, the sampled microbial population is a bacterial and/or fungal population.

Any method for analyzing nucleic acids may subsequently be employed. Preferably, the selected method must allow the structure and complexity of the bacterial or fungal community contained in the sample to be characterized.

In an example of an implementation of the method, the nucleic acid analysis consists in investigating specific genetic markers which allow the microbial population to be characterized, the set of markers constituting the genetic fingerprint.

A genetic fingerprint is obtained, for example, by amplification of specific nucleic acid fragments of the genome of the microbiological species, in particular by amplification of ribosomal RNA fragments, preferably from the 16S-23S intergenic space of the analyzed microorganisms, using a RISA technique described, for example, in Ranjard et al, 2001, Applied and Environmental Microbiology, October 2001, pp 4479-4487.

The following examples serve to illustrate certain specific implementations of the methods of the invention without in any way limiting its scope.

### KEY TO FIGURES

FIG. 1: FIG. 1 shows photographs of agarose gel in which nucleic acid extracts have been migrated.

A: Agarose gel of DNA extracted from the CH34Ralstonia metallidurans strain Tracks 1-4: calibration (500 ng, 250 ng, 124 ng, 62.5 ng respectively); tracks 5-7: chemical lysis; tracks 8-10: thermal lysis; tracks 11-13: mechanical lysis, 1600 rpm; tracks 14-16: mechanical lysis, 3000 rpm; tracks 17-19: BIO 101 kit.

B: Agarose gel of DNA extracted from the CFBP 1954 Bacillus polymixa strain. Tracks 1-4: calibration; tracks 5-7: chemical lysis; tracks 8-10: thermal lysis; tracks 11-13: mechanical lysis, 1600 rpm; tracks 14-16: mechanical lysis, 3000 rpm; tracks 17-19: BIO 101 kit.

C: Agarose gel of DNA extracted from the C7R12 Pseudomonas fluorescens strain. Tracks 1-4: calibration; tracks 5-7: chemical lysis; tracks 8-10: thermal lysis; tracks 11-13: mechanical lysis, 1600 rpm; tracks 14-16: mechanical lysis, 3000 rpm; tracks 17-19: BIO 101 kit.

D: Agarose gel of DNA extracted from the C58 Agrobacterium tumefaciens strain. Tracks 1-4: calibration; tracks 5-7: BIO 101 kit; tracks 8-10: chemical lysis; tracks 11-13: thermal lysis; tracks 14-16: mechanical lysis, 1600 rpm; tracks 17-19: mechanical lysis, 3000 rpm.

E: Agarose gel of DNA extracted from the Rhodococcus strain. 20 μl deposit. Tracks 1-4: calibration; tracks 5-7: chemical lysis; tracks 8-10: thermal lysis; tracks 11-13: mechanical lysis, 1600 rpm; tracks 14-16: mechanical lysis, 3000 rpm; tracks 17-19: BIO 101 kit.

FIG. 2: FIG. 2A is a histogram illustrating the quantities of DNA obtained for each strain with the various protocols: chemical, thermal, mechanical 1600 rpm, mechanical 3000 rpm and BIO 101 kit.

FIG. 2B is a histogram illustrating the quantities of DNA obtained for each protocol with the various strains. The bars represent the standard deviations.

Agrobacterium, Ralstonia, Bacillus, Pseudomonas and Rhodococcus

FIG. 3: FIG. 3A is a photograph of an agarose gel on which DNA extracted by a method combining two different lysis modes has been migrated:

Track M: size marker

Tracks 1-4: calibration

Tracks 5-7: Agrobacterium lyses, mechanical+chemical

Tracks 8-10: Agrobacterium lyses, chemical+thermal

Tracks 11-13: Ralstonia l lyses, mechanical+chemical

Tracks 14-16: Ralstonia lyses, chemical+thermal

Tracks 17-19: Bacillus, lyses mechanical+chemical

Tracks 20-22: Bacillus lyses, chemical+thermal

Tracks 23-25: Pseudomonas lyses, mechanical+chemical

Tracks 26-28: Pseudomonas lyses, chemical+thermal

Tracks 29-31: Rhodococcus lyses, mechanical+chemical

Tracks 32-34: Rhodococcus, chemical+thermal

Tracks 35-37: spores lyses, mechanical+chemical

Tracks 38-40: spores lyses, thermal+mechanical

FIG. 3B is a photograph of an agarose gel on which DNA extracted by a method combining the three different lysis modes has been migrated:

Tracks 1-4: calibration

Tracks 5-7: Agrobacterium

Tracks 8-10: Pseudomonas

Tracks 11-13: Rhodococcus

Tracks 14-16: spores

FIG. 4: FIG. 4 is an agarose gel on which DNA extracted from spores of Bacillus globigii has been migrated.

Track M: size marker

Tracks 1-3: calibration

Tracks 4-6: three repetitions of 30 μl deposits.

FIG. 5: FIG. 5 is a histogram illustrating the quantities of DNA obtained for each strain with the various protocols combining two or three lysis modes.

Mechanical+chemical; chemical+thermal;

Chemical+thermal+mechanical.

FIG. 6: FIG. 6 is an agarose gel on which DNA extracted from spores of Bacillus globigii have been migrated in a concentration of 10⁹ spores/ml, with deposits of 30 μl.

Track M: size marker; tracks 1-4; calibration: tracks 5-7: 3 deposits of 30 μl.

FIG. 7: FIG. 7A is an agarose gel on which DNA extracted from spores of Bacillus globigii, speywood strain, have been migrated. Deposits: 10 μl. Track M: size marker; tracks 1-3: calibration; tracks 4-6: no culture; tracks 7-9: +0.5 h in TS at 37° C.; tracks 10-12; 1 h in TS at 37° C.; tracks 13-15: +1.5 h in TS at 37° C.; tracks 16-19: +2 h in TS at 37° C.

FIG. 7B is an agarose gel on which 16S DNA from spores of Bacillus globigii, speywood strain, amplified with Qbiogene Taq polymerase, has been migrated. Deposits: 10 μl. Track M: size marker; tracks 1-3: +0.5 h in TS at 37° C.; tracks 4-6: no culture; track 7: negative control; track 8: positive control.

FIG. 8: FIG. 8A is an agarose gel on which DNA extracted from communities reconstituted at different concentrations have been migrated, with the extraction method of the invention combining the three lysis modes; deposits of 10 μl.

Track M: size marker; tracks 1-4: calibration; tracks 5-8: 6×10⁸ CFU/ml; tracks 9-12: 6×10⁷ CFU/ml; tracks 13-16: 6×10⁶ CFU/ml.

FIG. 8B is an agarose gel on which DNA extracted from communities reconstituted at different concentrations has been migrated using the extraction method of the invention combining the three lysis modes; 20 μl deposits.

Track M: size marker; tracks 1-4: calibration; tracks 5-8: 6×10⁵ CFU/ml; tracks 9-12: 6×10⁴ CFU/ml; tracks 13-16: 6×10³ CFU/ml; tracks 17-20: 6×10² CFU/ml.

FIG. 9: FIG. 9 is an agarose gel on which DNA extracted from spores and vegetative forms of Bacillus globigii (CIP 7718 strain and speywood strain) has been migrated, with the extraction method of the invention combining the three lysis methods; deposits of 20 [μl.

Track M: size marker; tracks 1-3: calibration; tracks 4-6: vegetative forms of strain CIP7718; tracks 7-9: sporulated forms of strain CIP7718; tracks 10-12: vegetative forms of speywood strain; tracks 13-15: sporulated forms of speywood strain.

### EXAMPLES

Five strains representative of large bacterial groups were used, as well as sporulated forms, in order to compare the extract yields obtained using a commercial nucleic acid extraction kit and various protocols based on the principles of chemical lysis, thermal lysis and mechanical lysis. After this first screening, any synergistic effect of the association of several protocols was tested, as well as optimization of the step for germination of sporulated forms. The extraction method of the invention was then carried out using bacterial communities termed “reconstituted” communities at different concentrations.

A/ Solutions, Stains, Culture Conditions and Titration

LB Medium:

Bacto-tryptone 10 g

NaCl 5 g

Yeast extract 10 g

H₂O qsp 1 1

+Agar 15 g/l

B King Medium:

| Biogelitone | 20 | g |
|---|---|---|
| Glycerol | 10 | ml |
| KH₂PO₄ | 1.5 | g |
| MgSO₄, 7H₂O | 1.5 | g |
| H₂O | qsp 1l | |
| +Agar | 15 | g/l |

Preparation of DNA Extraction Buffer, TES+2% SDS

Preparation with 0.01% Tween 20: TWEEN 20 1 ml+999 ml

Tris-HCl 1 M—pH 8

For 500 ml:

60.57 g Tris

+25 ml HCl to adjust to pH 8

+H₂O qsp 500 ml

Sterilization: Autoclave

Storage: ambient temperature.

20% SDS

For 500 ml:

100 g in about 400 ml H₂O then heat to dissolve

+H2O qsp 500 ml

Sterilization: filtration, but not necessary

Storage: ambient temperature.

1 M NaCl

For 500 ml:

29.22 g NaCl

+H₂O qsp 500 ml

Sterilization: Autoclave

Storage: ambient temperature.

EDTA 0.5 M—pH 8

For 500 ml: 12

93.085 g EDTA

+400 ml H₂O

+adjust to pH 8 with concentrated NaOH (EDTA soluble at pH 8)

+H₂O qsp 500 ml

Sterilization: Autoclave

Storage: ambient temperature.

Extraction Buffer (Final Concentrations):

TRIS HCl 100 mM pH 8, EDTA 100 mM pH 8, NaCl 100 mM, 2% SDS.

Thus! for 100 ml buffer: 10 ml of TRIS !M, 20 ml of EDTA 0.5 M, 10 ml of NaCl 1M, 10 ml of 20% SDS,+50 ml.

Potassium acetate 3M—pH 5.5

For 100 ml:

60 ml potassium acetate 5 M

+11.5 ml of 100% acetic acid

+H2O qsp 100 ml

Sterilization: filtration

Storage: ambient temperature.

Bacterial strains used are representative of the major bacterial types:
alpha-proteobacteria: Agrobacterium tumefaciens C58
beta-proteobacteria: Ralstonia metallidurans CH34
gamma-proteobacteria: Pseudomonas fluorescens C7R12
gram+low GC: Bacillus polymyxa CFBP 1954 (collection francaise des bactéries phytopathogènes)
gram+high GC: Rhodococcus rhodochrous ATCC 13898
spores of Bacillus globigii strain CIP 7718: 5 ml of suspension in water+0.01% TWEEN 20, in a concentration of 10⁹ spores/ml and used during extraction in a concentration of 10⁸ spores/ml.

The bacteria Ralstonia, Pseudomonas, Bacillus, Rhodococcus were cultivated in Luria-Bertoni (LB) medium, and Agrobacterium was cultivated in B King medium. At an optical density of 1 (600 nm), the suspensions, dilutions and calibrations of each strain were carried out in suitable media (LB agar and B King agar). The strains were titrated by counting colonies after incubating the dishes at 28° C. for 4 days. At this same optical density, 1 ml aliquots of cultures were removed, centrifuged (8000 g, 15 minutes) and frozen (−20° C.) for the future DNA extraction steps. This step prevented any lysis of bacterial cells in the culture medium during freezing.

The counts obtained are shown in Table 1 below. At an optical density of 1, at a wavelength of 600 nm, all of the strains were in the range 3.7×10⁸ to 1.6×10⁹ CFU/ml, a sufficient density for DNA to be extracted.

TABLE 1
Titration of strains

| Strains | CFU/ml | +/− | Standard deviation |
|---|---|---|---|
| Agrobacterium | 1.60 × 10⁹ | +/− | 0.49 × 10⁸ |
| tumefaciens C58 | | | |
| Bacillus polymyxa | 4.62 × 10⁸ | +/− | 7.07 × 10⁷ |
| CFBP 1954 | | | |
| Pseudomonas | 6.85 × 10⁸ | +/− | 1.20 × 10⁸ |
| fluorescens C7R12 | | | |
| Ralstonia | 3.76 × 10⁸ | +/− | 0.94 × 10⁷ |
| metallidurans CH34 | | | |
| Rhodococcus | 8.30 × 10⁸ | +/− | 0.52 × 10⁸ |
| rhodochrous | | | |
| ATCC13898 | | | |

B/ Extracting DNA from Strains
1/ Separate protocols

DNA from 5 bacterial strains and spores were extracted using the extraction protocols described in Example B. For each sample, 3 repetitions were carried out. The protocols used were based on the major DNA extraction principles routinely used in molecular ecology, namely (i) mechanical lysis based on the action of glass beads weakening bacterial membranes, (ii) chemical lysis based on the action of a detergent, sodium dodecyl sulfate (SDS), to weaken the membranes; and finally (iii) thermal lysis based on alternate hot and cold shocks, to burst the cells.

The mechanical lysis protocol comprises the following first steps:
start with a bacterial residue;
take up in 1 ml of TES extraction buffer (made with H₂O+0.01%TWEEN 20) (Tris-HCl 100 mM pH 8, EDTA 100 mM pH 8, NaCl 100 mM);
add 500 mg of washed 106 μm beads and 4×2mm diameter beads (in practice, weigh the beads into suitable tubes then add buffer+residue);
agitate the tubes in a mill at 1600 rpm for 30 seconds or 3000 rpm for 1 minute (support initially at −20° C.);
centrifuge at 14000 g, for 2 minutes (to separate out the beads) then recover the supernatant.

The thermal lysis protocol comprises the following first steps:
start with a bacterial residue;
take up in 1 ml of TES extraction buffer (made with H₂O+0.01%TWEEN 20) (Tris-HCl 100 mM pH 8, EDTA 100 mM pH 8, NaCl 100 mM);
3 cycles of heat shock:
liquid nitrogen (to freezing)→boiling water (3 minutes at 100° C.).

The chemical lysis protocol comprises the following first steps:
start with a bacterial residue;
take up in 1 ml of TES extraction buffer (made with H₂O+0.01% TWEEN 20) (Tris-HCl 100 mM pH 8, EDTA 100 mM pH 8, NaCl 100 mM, 2% SDS);
incubate at 70° C. for 30 minutes, vortexing for 10 seconds after 15 and 30 minutes.

The last steps are common to all protocols. They consist initially of adding K proteinase to denature the proteins, then incubating with potassium acetate over ice to precipitate and eliminate the proteins after centrifuging (14000 g, 5 min). Finally, the DNA is precipitated using cold isopropanol (v/v).

The common steps of the protocol are listed below:
add K proteinase: 20 μl of a 10 mg/l mother liquor;
incubate 30 minutes between 37° C. and 50° C.;
add 1/10 of volume (about 100 μl) of 3M potassium acetate, pH 5.5;
incubate 10 minutes over ice;
centrifuge at 14000 g for 5 minutes and recover the supernatant in a 2 ml tube;
add 1 volume of isopropanol at −20° C.;
30 minutes at −20° C.;
centrifuge at 13000 rpm for 30 minutes;
eliminate the supernatant with care, using a pipette;
wash the residue with 70° ethanol (200 μl) at −20° C. (without taking the DNA into suspension). Centrifuge for 5 minutes at 13000 rpm;
eliminate alcohol and allow the residue to dry for 30 minutes at ambient temperature (or in an oven) until there are no more traces of alcohol on the tube walls;
take up the residue in 100 μl of H₂O.

The protocols for preparing the various buffers were described above.

The FASTDNA® KIT (referred to as KIT BIO101 in the text) commercial DNA extraction kit is a combination of mechanical and chemical lysis. The protocol described by the manufacturer, Qbiogene, is described in the technical note provided with the kit.

The DNA extracted from each bacterial strain is quantified on gel by comparison with the calf thymus DNA calibration curve. 10 μl of DNA extract are deposited on 1% agarose gel. A calf thymus DNA calibration curve corresponding to 500 ng DNA/10 μl, 250 ng DNA/10 μl, 125 ng DNA/10 μl, 62.5 ng DNA/10 μl is also deposited. After migration and staining with ethidium bromide, the gel is processed with an image analyzer to allow a calibration curve to be calculated and finally, to allow the quantities of DNA extracted for each strain to be determined.

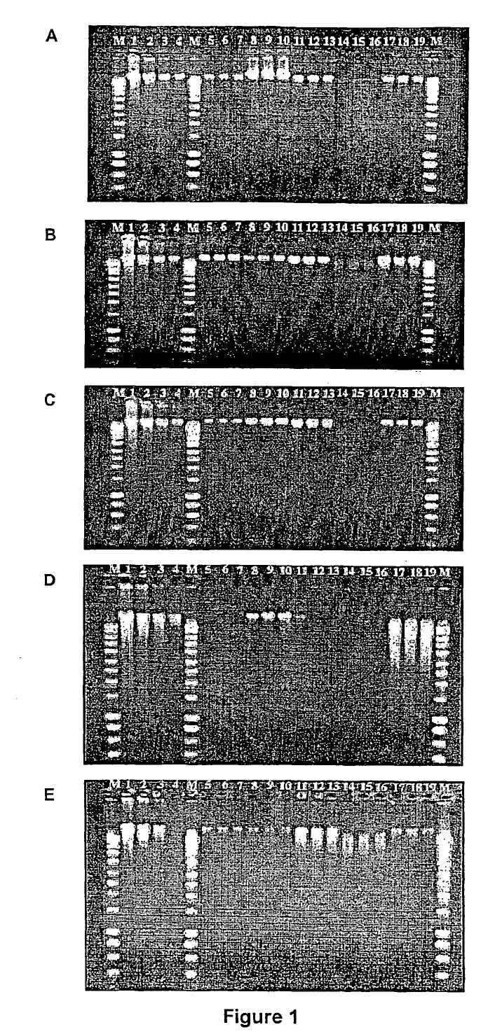

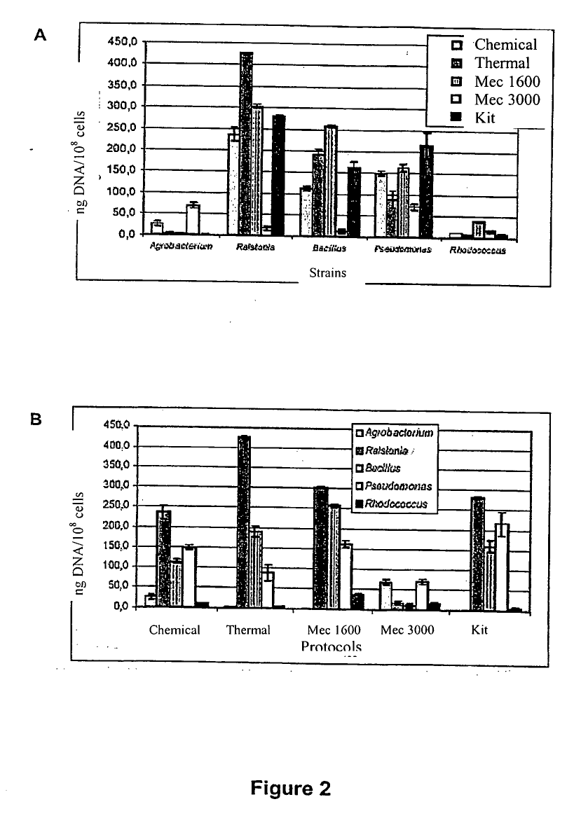

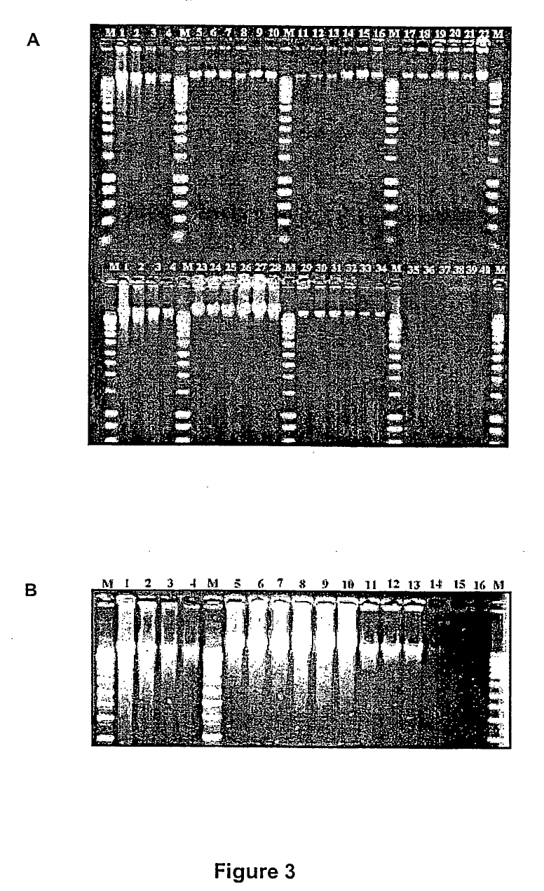

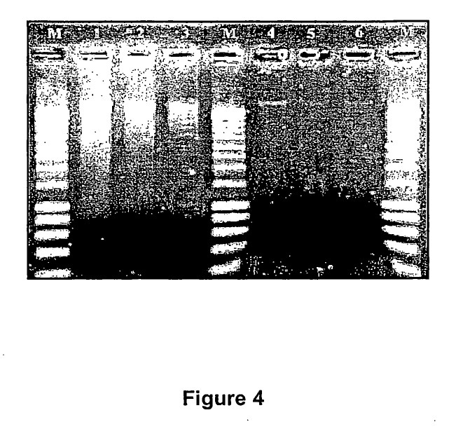

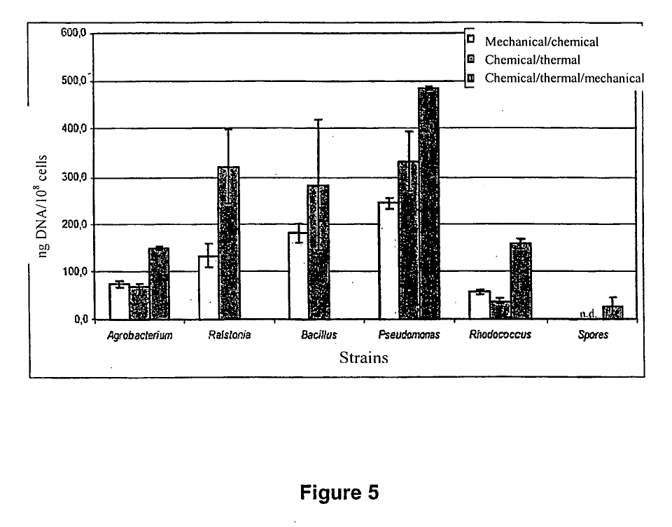

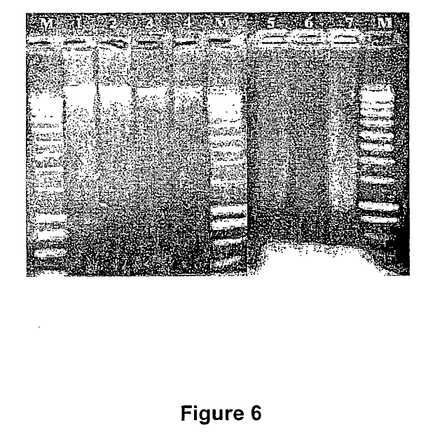

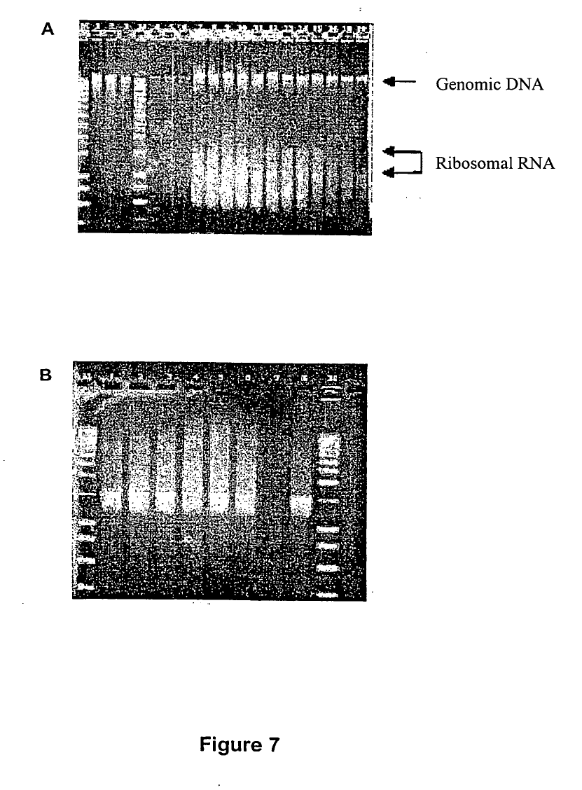

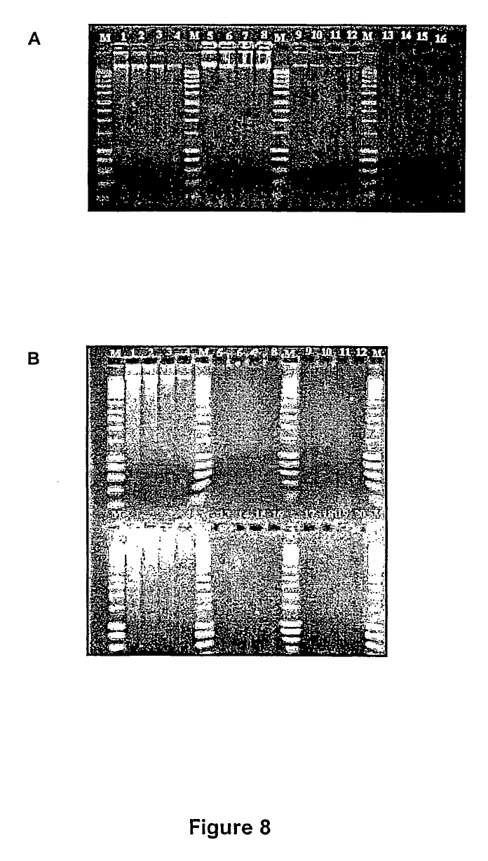

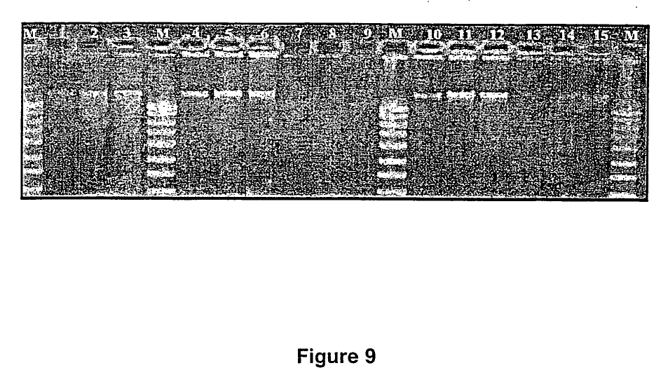

FIGS. 1A to 1E show deposits of 10 μl of DNA for Agrobacterium, Pseudomonas, Bacillus, Ralstonia and 20 μl for Rhodococcus respectively. The gel corresponding to the spores was not present as no DNA could be detected visually. The photos clearly show (i) the negative effect of mechanical lysis at 3000 rpm for 1 minute on the integrity of the extracted DNA and (ii) the extreme heterogeneity of the DNA yields when comparing the protocols. This observation is confirmed by the digital processing of the image. The quantities of the DNA, standardized to 10⁸ CFU/ml, are shown in Table 2 below and presented, as a function of the strains in FIG. 2A and as a function of the protocols in FIG. 2B. The quantities of DNA are in the range 0.9 to 425 ng of DNA/10⁸ CFU.

TABLE 2
Quantity of DNA for 10⁸ CFU extracted from each strain with the various protocols:

| NgDNA/10⁸ CFU | | Agrobacterium | Agrobacterium | Ralstonia | Bacillus | Pseudomonas |
|---|---|---|---|---|---|---|
| Chemical | Mean | 26.0 | 236.1 | 113.3 | 149.4 | 10.8 |
| | Std | 5.4 | 16.4 | 5.7 | 5.8 | 0.4 |
| | dev | | | | | |
| Thermal | Mean | 3.5 | 425.2 | 190.7 | 89.0 | 6.6 |
| | Std | 1.8 | 3.2 | 12.9 | 20.4 | 0.3 |
| | dev | | | | | |
| Mec 1600 | Mean | 1.6 | 302.4 | 257.5 | 162.7 | 38.0 |
| | Std | 1.3 | 4.1 | 3.9 | 9.0 | 0.9 |
| | dev | | | | | |
| Mec 3000 | Mean | 69.4 | 17.8 | 12.0 | 71.6 | 16.1 |
| | Std | 7.0 | 4.0 | 4.5 | 7.1 | 2.8 |
| | dev | | | | | |
| Kit | Mean | 0.9 | 280.5 | 160.6 | 216.2 | 7.7 |
| | Std | 0.3 | 2.8 | 14.8 | 29.0 | 2.0 |
| | dev | | | | | |

The principal teachings of these results are (i) none of the protocols seem to be suitable for equivalent extraction, in terms of yield, for all of the tested strains, and (ii) apart from the kit, mechanical lysis at 1600 rpm for 30 seconds, chemical lysis and thermal lysis appear to produce the best results.

2/Synergy of Lysis Protocols on Extraction Yields

a/ Synergy of Two Protocols

To increase the quantities of DNA extracted, and also to rank the differences between the strains, a possible synergistic effect of the extraction protocols was evaluated. To this end, the protocols which produced the most significant results were combined independently.

The chemical lysis/mechanical lysis at 1600 rpm for 30 seconds and chemical lysis/thermal lysis combinations were tested.

FIG. 3A corresponds to deposits of 10 μl of DNA for Agrobacterium, Pseudomonas, Bacillus, Ralstonia and 20 μl for Rhodococcus and spores. However, no DNA was observed for the spores, as indicated on tracks 35 to 40. The quantities given in FIG. 5 are in the range 37.4 to 332.4 ng of DNA/10⁸ CFU. A synergistic effect was observed for the Agrobacterium and Rhodococcus strains, while no significant differences were observed between the two different combinations.

For strains of Bacillus, Ralstonia and Pseudomonas, a regularization of the quantity of DNA was observed for the two combinations. This quantity was higher for the chemical lysis+thermal lysis combination, but there was a large variability between the repetitions for a single strain (see the standard deviations in FIG. 5).

b/ Synergy of Combination of Three Lysis Modes

A combination of three lysis protocols was also tested to evaluate any synergistic effect, i.e. the combination of chemical lysis then thermal lysis and finally mechanical lysis, with a step preceding K proteinase (weakening of cell membranes) and a rate of 1600 rpm for I minute. The detailed protocol for this synergy is presented below:
start with a bacterial residue;
take up in 1 ml of TES extraction buffer (made with H₂O+0.01% TWEEN 20)+2% SDS (Tris-HCl 100 mM pH 8, EDTA 100 mM pH 8, NaCl 100 mM, 2% SDS);
add 500 mg of washed 106 μm beads and 4×2mm diameter beads (in practice, weigh the beads into suitable tubes then add buffer+residue);
add K proteinase: 20 μl of a 10 mg/ml mother solution;
incubate 30 minutes at 37° C.;
incubate at 65° C. for 30 minutes by vortexing 10 seconds after 15 and 30 minutes (chemical);
3 cycles of heat shock:
liquid nitrogen (until freezing)→boiling water (3 minutes at 100° C.) (thermal).
agitate the tubes in a mill at 1600 rpm for I minute (support initially at −20° C.) (mechanical);
centrifuge at 14000 g, for 1 minute and recover the supernatant into a 2 ml tube.
add 1/10 by volume (about 100 μl) of 3M potassium acetate, pH 5.5;
incubate 10 minutes on ice;
centrifuge at 14000g for 5 minutes and recover the supernatant in a 2 ml tube;
add 1 volume of isopropanol at −20° C.;
incubate for 30 minutes at −20° C.;
centrifuge at 13000 rpm for 30 minutes;
eliminate supernatant carefully using a pipette;
wash the residue with 70° ethanol (200 μl) at −20° C. (without taking the DNA into suspension). Centrifuge for 5 minutes at 13000 rpm;
eliminate alcohol and allow the residue to dry for 30 minutes at ambient temperature (or in an oven) until there are no more traces of alcohol on the tube walls;
take up the residue in 100 μl of H₂O.

This combination of three lysis protocols was used to lyse spores of Bacillus globigii and two strains (Agrobacterium and Rhodococcus) for which the extraction yields were the lowest, as well as the Pseudomonas strain, used as an extraction control.

FIG. 3B represents deposits of 10 μl of DNA for Pseudomonas, Rhodococcus, Agrobacterium and spores. The results for the quantities of DNA are shown in FIG. 5. The poor nature of the standard deviations is immediately noticeable, but note also the strong synergistic effect of this technique. The quantities of DNA are respectively doubled and tripled with respect to the synergistic effect of the two lysis modes, for Agrobacterium and Rhodococcus. Further, this synergy allowed DNA to be extracted from spores (Bacillus globigii strain CIP 7718) in an amount of 30 ng DNA/10⁸ spores. The deposit of 30 μl (FIG. 4) of DNA extracted from spores allowed high molecular weight DNA to be visualized.

With the aim of confirming these results for spores, the extraction protocol combining the three lysis modes was carried out on 10⁹ spores. FIG. 6 shows the non negligible quantity of DNA extracted from spores. After quantification, the extracted DNA was estimated to be 1080 ng for 10⁹ spores, i.e. a yield of 24% efficiency for extraction.

The combination of chemical lysis, thermal lysis and mechanical lysis is thus suitable for the extracting nucleic acids from microorganisms representing the major phylums and produces a synergistic effect on the extraction yields obtained. The principal advantages of this synergy are thus:
(i) a quantity of extracted DNA from all strains which is sufficient for amplification by PCR;
(ii) good reproducibility, and finally
(iii) the possibility of extracting DNA from spores, which are highly resistant elements potentially present in high densities in air.
   C/ Extraction Yield

The extraction yields were calculated with the size of the genome of the strains used, obtained from publicly available databases (http://www.ncbi.nlm.nih.gov/PMGifs/Genomes/eub_g.html) and knowing that 1 Mb corresponds to 1×10⁻¹⁵ g DNA. Knowing the quantities of DNA extracted in ng/10⁸ CFU, it is thus possible to determine yields which are in the range 10% for spores and 75% for Pseudomonas (with the combination of three lysis protocols). However, the mean of these yields, if that obtained for spores is ignored, is around 50%.

The DNA extraction yields obtained by combining 2 or 3 lysis modes and estimated for each strain with respect to the genome size are indicated in Table 3 below:

TABLE 3
DNA extraction yield, estimated for each strain with
respect to genome size

| | Genome sizes | Ng DNA/10⁸ | |
|---|---|---|---|
| Strains | (Mb) | CFU | Yield (%) |
| Agrobacterium | 5.66 | 149.7 | 26.5 |
| tumefaciens C58* | | | |
| Bacillus polymyxa | 4.5 | 282.5 | 62.8 |
| CFBP 1954** | | | |
| Pseudomonas | 6.47 | 484.4 | 74.8 |
| fluorescens C7R12* | | | |
| Ralstonia | 6.76 | 321.5 | 47.6 |
| metallidurans | | | |
| CH34** | | | |
| Rhodococcus sp.* | 6.5 | 157.8 | 24.6 |
| Spores 10⁸ cells* | 4.5 | 47.7 | 10.4 |
| Spores 10⁸ cells* | 4.5 | 1080/10⁹ cells | 24 |

**quantity obtained with synergy of 2 protocols
*quantity obtained with synergy of 3 protocols.

D/ Extraction from “Reconstituted Communities”

To determine the limit of sensitivity of the extraction method of the invention, DNA from “reconstituted” communities was extracted at different concentrations using the extraction method of the invention combining three lysis types.

To this end, 10⁸ CFU from 5 bacterial strains as well as sporulated forms (Bacillus globigii strain CIP 7718) was taken up into suspension in 1 millilitre of solution. This suspension at a concentration of 6×10⁸ CFU/ml acted as the mother solution for one tenth dilutions to 6×10² CFU/ml. Four repetitions per dilution were used to extract DNA. The results are presented in FIGS. 8A and 8B with deposits of 10 μl for concentrations of 6×10⁸ to 6×10⁶ CFU/ml and deposits of 20 μl of 6×10⁵ to 6×10². The quantity of DNA obtained for 6×10⁸ was about 3000 ng with a yield of 82% for a mean genome size of 5.5 Mb. At a density of 6×10⁷ CFU/ml, the quantity of DNA reduced by a factor of about 10 to reach 400 ng. For high dilutions (6×10⁶ to 6×10² CFU/ml), the extracted DNA was not visible. A theoretical calculation resulted in a concentration of 4 ng of DNA for 10 μl deposited at a cell density of 6×10⁶ CFU/ml. Such a quantity is not visible on agarose gel with ethidium bromide staining, which explains the results. The extraction method combining the three lysis modes can thus extract DNA in a highly satisfactory manner from reconstituted communities with relatively high yields.

E/ DNA Extraction Test Using Spores of another strain of Bacillus globigii: the Speywood Strain

The results obtained show that the spores are reluctant to be lysed. Even using the extraction method of the invention, the DNA extraction yields remain low. To improve these yields, a protocol including a prior step of taking up the sporulated forms into culture in a TS liquid medium (soy trypticase) for 2 hours at 37° C. was tested. This incubation allows the sporulated forms to change to vegetative forms. The sample DNA was then extracted using the extraction method of the invention. This protocol was tested on two strains of Bacillus globigii: speywood (can be turned into an aerosol) and CIP 7718 (cannot be turned into an aerosol).

The DNA obtained from the vegetative and sporulated forms of the two strains of Bacillus globigii was deposited on gel and the results are shown in FIG. 9. The quantities corresponding to these deposits are given in Table 4 below.

TABLE 4

| | | Qy DNA | Mean | | |
|---|---|---|---|---|---|
| | | (ng/10⁸ | (ng/10⁸ | Theoretical | Sporulation |
| | Strains | spores) | spores) | yield (%) | effect (%) |
| Vegetative | CIP1 | 135.6 | 193.3 | 43.0 | 3.3 |
| forms | CIP2 | 210.9 | | | |
| (cannot be | CIP3 | 233.5 | | | |
| turned into | | | | | |
| an aerosol) | | | | | |
| Sporulated | CIP4 | 12.0 | 6.3 | 1.4 | |
| forms | CIP5 | 7.4 | | | |
| (cannot be | CIP6 | −0.6 | | | |
| turned into | | | | | |
| an aerosol) | | | | | |
| Vegetative | Spey 1 | 106.6 | 172.9 | 38.4 | 16.1 |
| forms | Spey 2 | 224.1 | | | |
| (can be | Spey 3 | 188.0 | | | |
| turned into | | | | | |
| an aerosol) | | | | | |
| Sporulated | Spey 4 | 21.4 | 27.8 | 6.2 | |
| forms | Spey 5 | 31.1 | | | |
| (can be | Spey 6 | 30.9 | | | |
| turned into | | | | | |
| an aerosol) | | | | | |

The results obtained show that the change from the sporulated form to the vegetative form allows an increase in the quantity of extracted DNA regardless of the strain under consideration (from 6 to 193 ng DNA/10⁸ spores of the CIP strain and 28 to 173 ng of DNA/10⁸ spores for the speywood strain).

Considering that the time to generate the majority of the environmental bacteria is more than 1 hour, the incubation step of 2 hours at 37° C. should not modify the composition and density of the bacterial community in the samples. This step could thus prove of interest for the effective extraction of DNA from spores contained in air samples.

F/ Effect of Culturing on Extraction Yield and Amplification of DNA from Sporulated Forms.

Since sporulated forms could potentially represent a large part of the biological air background, it appears to be important to investigate a means for improving the yield. During the first step, the effect of germination on the yield for extracting DNA from samples of sporulated forms was tested by incubating the spores from two strains, CIP7718 and speywood, from Bacillus globigii at 37° C. in a culture medium (TS) for 2 h. This incubation step was intended to induce passage from the sporulated forms to the vegetative forms which were easier to lyse. The cells were then lysed and the extracted DNA deposited on a gel. The results obtained showed that for the two test strains, the incubation step significantly improved the DNA extraction yield from sporulated forms.

Assuming that sporulated forms could represent a large fraction of the biological background in air, this incubation step thus appears to be necessary. However, it must be of a duration which is sufficiently short to prevent the growth of bacterial populations in the vegetative form, which are also present in the samples. This is why spore germination kinetics were studied to determine the minimum incubation time sufficient to improve the DNA extraction yield. To this end, the sporulated forms of the speywood strain of Bacillus globigii were incubated at 37° C. in TS medium for 0.5; 1; 1.5; and 2 h.

DNA was then extracted using the 3-way synergy protocol and deposited on gel (FIG. 7A). The results obtained show that 30 minutes incubation suffices to significantly improve the DNA extraction yield from sporulated forms. This incubation is sufficiently short for no modification of the cell density of the samples to occur and for the relative balance of the various bacterial populations not to be biased.

Subsequently, we carried out PCR amplification of the 16S gene of the DNA extracted from sporulated forms after 0 and 0.5 hours incubation. The results obtained are shown in FIG. 7B. The profiles show that the amplification was effective for the sporulated forms after 0 and 0.5 hours of culture in TS medium at 37° C.

However, it appeared that this incubation step significantly improved the yield and the reproducibility of the extraction and amplification of DNA from spores.

G/ Analysis of DNA Extracts from Microbial Populations Sampled from Air.

To study microbial communities sampled from air using molecular biological tools, the nucleic acid extracts obtained using the extraction method of the invention were analyzed by carrying out genetic fingerprinting of the population using the RISA (ribosomal intergenic spacer analysis) technique as described by Ranjard et al, 2001, Applied and Environmental Microbiology, pp 4479-4487, or T-RFLP (terminal restriction fragment length polymorphism) (Moesenieder et al, 2001, Journal of microbiological methods, vol 44 (2) pp 159-172; Saka et al, Soil Science and Plant Nutrition, vol 47 (4) 773-778; Urakawa et al, 2000, MEPS, 220: 47-57; Marsh 1999, Current opinions in Microbiology, vol 2 (3), 323-327.

1. A method for extracting nucleic acids from microorganisms, comprising:
a. successive implementation of the following three lysis steps in any order:
i) carrying out a chemical lysis of microorganisms, comprising bringing microorganisms into contact with a lysis solution containing a detergent;
ii) carrying out a heat shock lysis, comprising incubating an extract at a first temperature below 0° C., immediately followed by incubating the extract at a second temperature of at least 95° C.; and
iii) carrying out a mechanical lysis, comprising agitating an extract in the presence of beads.
b. precipitating a membrane and protein debris;
c. recovering a supernatant containing the nucleic acids; and
d. precipitating the nucleic acids and eliminating the supernatant;

2. The method according to claim 1, wherein the second temperature in heat shock lysis, in step (a) (ii), is carried out at about 100° C.

3. The method according to claim 1, comprising further, after step d, taking the nucleic acids up into solution in a buffer.

4. The method according to claim 1, wherein the three lysis steps defined in (a) are carried out in the following order: (i) chemical lysis, (ii) heat shock lysis and (iii) mechanical lysis.

5. The method according to claim 1, wherein the detergent in step (a) (i) is sodium dodecyl sulfate (SDS).

6. The method according to claim 5, wherein the detergent is SDS in a concentration in the range 1% to 4%.

7. The method according claim 1, wherein the lysis in step (a) (ii) comprises incubating the extract at a temperature of about −70° C. immediately followed by incubating the extract at a temperature of at least 95° C.

8. The method according to claim 7, wherein the −70° C. temperature is carried out in liquid nitrogen.

9. The method according to claim 7, wherein the second temperature is carried out at about 100° C.

10. The method according to claim 9, wherein the second temperature is carried out in boiling water.

11. The method according to claim 1, wherein the mechanical lysis, in step (a) (iii), comprises agitation in a mill in the presence of beads with a diameter in the range 50 μm to 200 μm.

12. The method according to claim 11, wherein the beads have a diameter of about 100 μm.

13. The method according to claim 1, wherein the microorganisms are bacteria and/or fungi.

14. The method according to claim 1, wherein the microorganisms are spores of bacteria and/or fungi.

15. The method according to claim 1, wherein the three lysis steps are preceded by a step for culturing spores, comprising culturing spores in a medium for initiating germination.

16. The method according claim 1, wherein the microorganisms are sampled from air.

17. A method for analyzing a microbial population of an environment, comprising:
removing a sample of the microbial population from the environment and taking the sample up into suspension in a solution;
extracting nucleic acids from the microbial population in suspension using the method defined in claim 1;
analyzing the extracted nucleic acids.

18. A method for analyzing a microbial population of an environment, comprising:
removing a sample of a microbial population from the environment and taking the sample up into suspension in a solution;
extracting nucleic acids from the microbial population in suspension using the method defined in claim 15;
analyzing the extracted nucleic acids

19. The method according to claim 17, wherein the microbial population to be analyzed is sampled from air.

20. The method according to claim 17, wherein the microbial population is a bacterial and/or fungal population.

21. The method according to claim 17, wherein the analysis of the extracted nucleic acids comprises using a set of genetic markers which can characterize the microbial population, the set of genetic markers constituting a genetic fingerprint.

22. The method according to claim 21, wherein the genetic fingerprint is obtained by amplifying nucleic acid fragments specific to a genome of microbial species.

23. The method according to claim 22 wherein the genetic fingerprint is obtained by amplifying ribosomal RNA fragments of microbial species.

24. The method according to claim 23, wherein the ribosomal RNA fragments are from a 16S-23S intergenic space of the analyzed microbial species.
